(19) United States
(12) Patent Application Publication
(10) Pub. No.: US 2025/0027023 A1
(43) Pub. Date: Jan. 23, 2025

(54) MULTIFUNCTIONAL MICROBIAL UNIT CULTURE APPARATUS

(71) Applicant: MYCEL INC., Gunpo-si (KR)

(72) Inventor: Myeong Jun KIM, Suwon-si (KR)

(21) Appl. No.: 18/900,964

(22) Filed: Sep. 30, 2024

Related U.S. Application Data
(63) Continuation of application No. PCT/KR2022/016330, filed on Oct. 25, 2022.

(30) Foreign Application Priority Data
Dec. 29, 2021 (KR) 10-2021-0191667
Sep. 30, 2022 (KR) 10-2022-0124879

(51) Int. Cl. C12M 1/00; C12M 1/34; C12M 3/00

(52) U.S. Cl. CPC C12M 29/24; C12M 23/40; C12M 23/44; C12M 41/12

(57) ABSTRACT

The multifunctional microbial unit culture apparatus according to an embodiment of the present invention includes a main module that generates a mixed gas by mixing carbon dioxide and oxygen and controls the temperature and humidity of the mixed gas, and a cultivation module that includes a cultivation unit for cultivating microorganisms using the mixed gas supplied from the main module, wherein the mixed gas can circulate between the main module and the cultivation module.

### TECHNICAL FIELD

The present invention relates to a multifunctional microbial unit culture apparatus, and more particularly to a multifunctional microbial unit culture apparatus capable of supplying a mixed gas containing oxygen at an appropriate temperature and humidity to a culture unit. Through this, humidification process is carried out in accordance with the state of the cooled or heated mixed gas, enabling control of humidity and temperature within the cultivation module while preventing condensation. Additionally, the apparatus prevents the differentiation of mushrooms.

### BACKGROUND ART

In general, mushroom cultivation using fungi is a technology, labor, and capital-intensive industry. Mushrooms are produced in temporary or permanent cultivation facilities and require sophisticated environmental management as a crop demanding advanced techniques.

Fungal mycelia typically require precise control of nutrients, temperature, humidity, pressure, light, carbon dioxide and oxygen concentrations for cultivation. If even one of these conditions is not met, differentiation into mushrooms may be difficult. Additionally, fungal mycelia are aerobic and exchange oxygen and carbon dioxide through respiration from the moment cultivation begins. Without adequate oxygen supply, differentiation into mushrooms becomes challenging, the rate of deformed mushrooms increases, and the amount of mycelial growth is proportional to respiration rate. Therefore, frequent ventilation is necessary to control suitable environmental conditions for the target fungi until mushroom harvest. Ventilation methods and environmental control systems must be appropriately set according to the growth stage to ensure proper development.

Manufacturing fungal biomats requires a significantly higher concentration of carbon dioxide, along with high temperature and humidity, is required compared to the environment used for mushroom cultivation. For example, 5-7% carbon dioxide, 32-38° C. temperatures, and 99% humidity are required. This prevents the fungal mycelia from differentiating into mushrooms and forming spores. The high temperature also accelerates tissue activity to increase cultivation speed.

In the conventional method of producing fungal biomats, cultivation is conducted in large-volume cultivation chambers or room-type facilities, making it difficult to precisely control the cultivation environment. For example, in the case of heating-type humidifiers, ultrasonic humidifiers, or centrifugal humidifiers, water is supplied as fine particles; however, depending on the size of the water molecules, certain areas may become excessively humid, while others may dry out. In other words, the level of humidification may not be uniform.

Additionally, to provide air at the target temperature, the cooling or heating circulation methods generally include top discharge or bottom discharge systems. However, due to convection, the larger the volume, the more difficult it becomes to maintain a uniform temperature. Furthermore, when humidified air is heated or cooled, the relative humidity may become higher or lower than the target level. During the cooling process, condensation or freezing may cause water droplets to clump together or pool, which can interfere with the cultivation of the mycelium.

Therefore, in the cultivation of mycelium, there is a need for the development of a mycelium cultivation apparatus capable of precise environmental control. Such an apparatus should allow for cultivation at a controlled temperature, maintaining the target relative humidity without the formation of condensation or dripping water.

### DISCLOSURE

### Technical problems

An embodiment of the present invention provides a multifunctional microbial unit cultivation apparatus capable of supplying a mixed gas containing oxygen to the cultivation chamber at an appropriate temperature and humidity. This allows the humidification process to be conducted in accordance with the state of the cooled or heated mixed gas, enabling precise control of humidity and temperature within the cultivation module, thereby preventing condensation. Furthermore, the apparatus prevents the differentiation of mushrooms and promotes the growth of mycelium, leading to the formation of a fungal biomat.

The problems to be solved by the present invention are not limited to those mentioned above, and other problems not mentioned will be clearly understood by those skilled in the art from the following description.

### Technical Solution

The multifunctional microbial unit cultivation apparatus according to an embodiment of the present invention includes a main module that generates a mixed gas by mixing carbon dioxide and oxygen, and regulates the temperature and humidity of the mixed gas. The apparatus also includes a cultivation module that utilizes the mixed gas supplied from the main module to cultivate microorganisms. The mixed gas can be circulated between the main module and the cultivation module.

According to one aspect, the main module includes a housing that comprises a humidification unit containing multiple pipes and a heat exchange unit designed to facilitate heat exchange using a flowing fluid. The housing is equipped with a gas mixing unit that mixes carbon dioxide and oxygen to generate a mixed gas, a temperature control unit that regulates the temperature of the mixed gas, and a humidity control unit that adjusts the humidity of the mixed gas. The mixed gas, conditioned for temperature and humidity by the temperature control unit and the humidity control unit, can then be supplied to the cultivation module.

According to one aspect, the main module and the cultivation module may have a stacked configuration.

According to one aspect, the cultivation module may include a case, and further comprises an intake unit provided within the case for directing the mixed gas supplied from the main module to the cultivation section.

According to one aspect, the intake unit is positioned on both sides of the cultivation section within the case. The intake unit may include a velocity reduction member for reducing airflow velocity, a blow fan for circulating air, and a rail member on which the blow fan is movably mounted.

According to one aspect, the cultivation module may further include an eliminator, which is provided on one side of the cultivation section within the case, to prevent the scattering of water droplets contained in the mixed gas.

According to one aspect, the eliminator may include: a support fixed in the case; and a scattering prevention unit provided in a multi-layer structure on the support to prevent scattering of passing water droplets.

According to one aspect, the eliminator may include an inlet through which the mixed gas supplied from the main module is introduced, an outlet that discharges the mixed gas into the cultivation section, and a connecting passage that links the inlet and the outlet. The outlet may be positioned below the inlet, and the angle formed by the connecting passage may range from 30 to 60 degrees.

According to one aspect, the inlet and outlet may be arranged in a hexagonal configuration.

According to one aspect, the case includes a lower case member and an upper case member detachably coupled to the lower case member. One of the lower case member and the upper case member may be provided with a locking member, and the other may be provided with a locking receiver member, such that coupling or separation of the upper case member to/from the lower case member can be achieved by engagement or disengagement of the locking member and the locking receiver member.

According to one aspect, a V-shaped loop for collecting respiratory water generated during respiration of the microorganisms may be provided on the back surface of the upper case member.

According to one aspect, the cultivation section may consist of a plurality of cultivation vessels arranged in a parallel configuration.

### Advantageous Effects

According to an embodiment of the present invention, a mixed gas containing oxygen can be supplied to the cultivation section at an appropriate temperature and humidity. This enables the humidification process to be adjusted in accordance with the state of the cooled or heated mixed gas, thereby allowing precise control of humidity and temperature within the cultivation module, preventing condensation. Additionally, it prevents the differentiation of mushrooms and promotes the growth of mycelium, enabling the formation of a fungal biomats.

### BRIEF DESCRIPTION OF DRAWINGS

FIG. 1 is a schematic view showing the configuration of a multifunctional microbial unit culture apparatus according to an embodiment of the present invention.

FIG. 2 is a schematic view showing the internal configuration of the cultivation module shown in FIG. 1.

FIG. 3 is a perspective view of the cultivation module shown in FIG. 2.

FIG. 4 is another perspective view of the cultivation module of FIG. 3 viewed from a different direction.

FIG. 5 is a perspective view showing the internal configuration of the cultivation module shown in FIG. 2.

FIG. 6 is a plan view of FIG. 5.

FIG. 7 is a view for explaining the intake unit of the culture module shown in FIG. 5.

FIG. 8 is a side view of FIG. 7 for explaining the movement structure of the blow fan.

FIG. 9 is a perspective view of the eliminator shown in FIG. 5.

FIG. 10 is a cross-sectional view of FIG. 9.

FIG. 11 is a view for explaining the configuration of the loop part provided in the upper case member among the cases shown in FIG. 3.

### BEST MODE FOR INVENTION

The present invention relates to a multifunctional microbial unit cultivation apparatus capable of supplying a mixed gas containing oxygen to the cultivation section at an appropriate temperature and humidity. This capability enables the humidification process to be effectively managed based on the state of the cooled or heated mixed gas, preventing the occurrence of condensation within the cultivation module where humidity and temperature control takes place. Furthermore, it also prevents the differentiation of mushrooms. The invention includes a main module that generates a mixed gas by combining carbon dioxide and oxygen and regulates the temperature and humidity of the mixed gas, along with a cultivation module that utilizes the mixed gas supplied from the main module to cultivate microorganisms. The mixed gas may circulate between the main module and the cultivation module.

### Mode for Invention

The advantages and/or features of the present invention, as well as the methods for achieving such advantages and/or features, will become apparent through a detailed description of the embodiments referenced in conjunction with the accompanying drawings. However, the present invention is not limited to the embodiments disclosed herein but may be implemented in various forms. The disclosed embodiments are provided for the purpose of ensuring that the disclosure of the present invention is complete and to fully inform those skilled in the art regarding the scope of the invention. The present invention is defined solely by the scope of the claims. Throughout the specification, like reference numerals refer to like components.

Below, embodiments of the present invention will be described in detail with reference to the accompanying drawings.

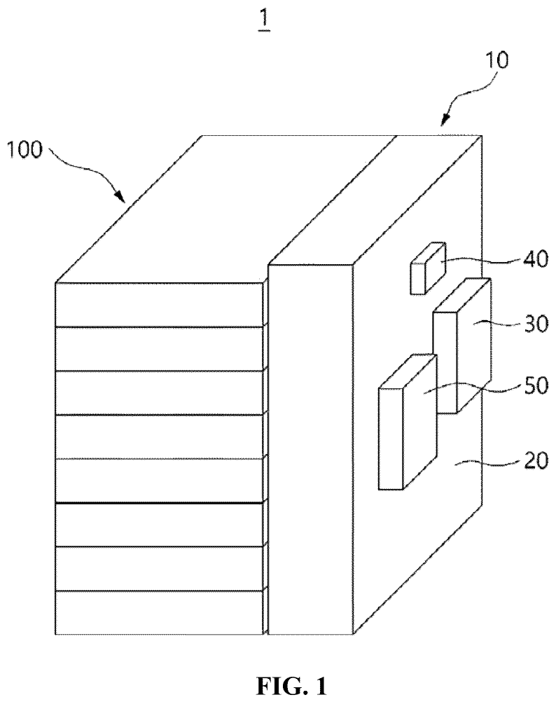

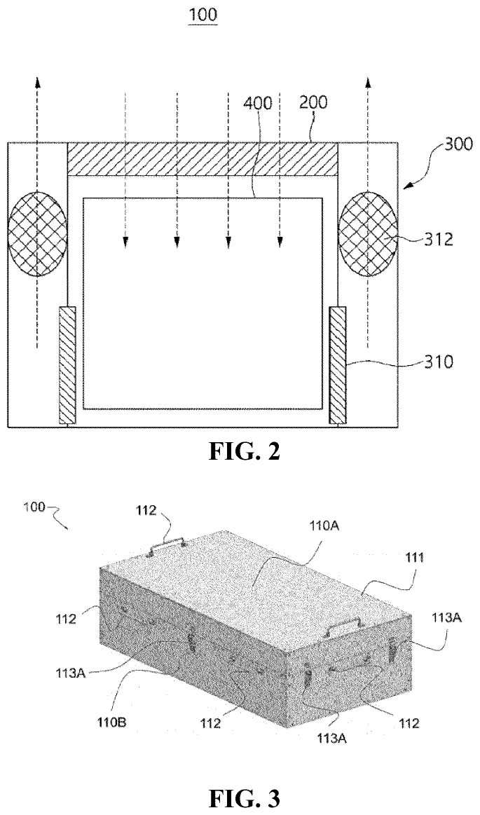

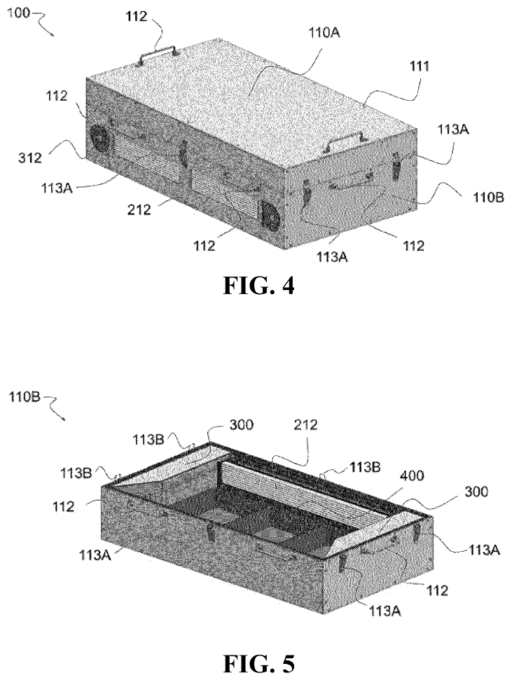

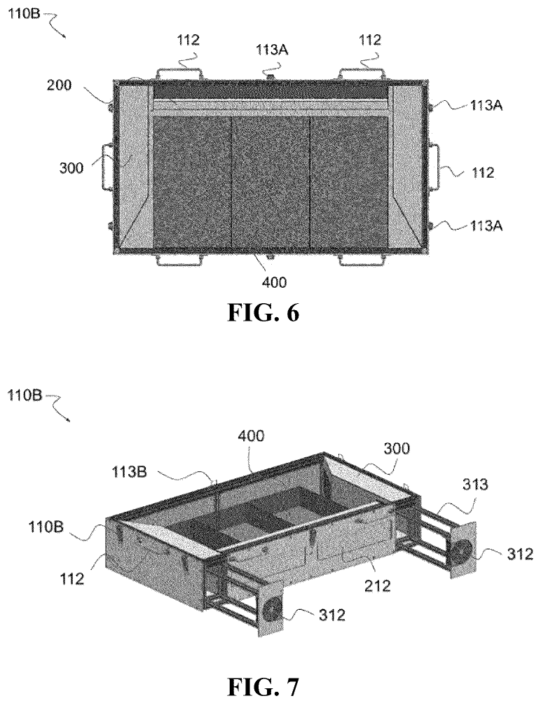

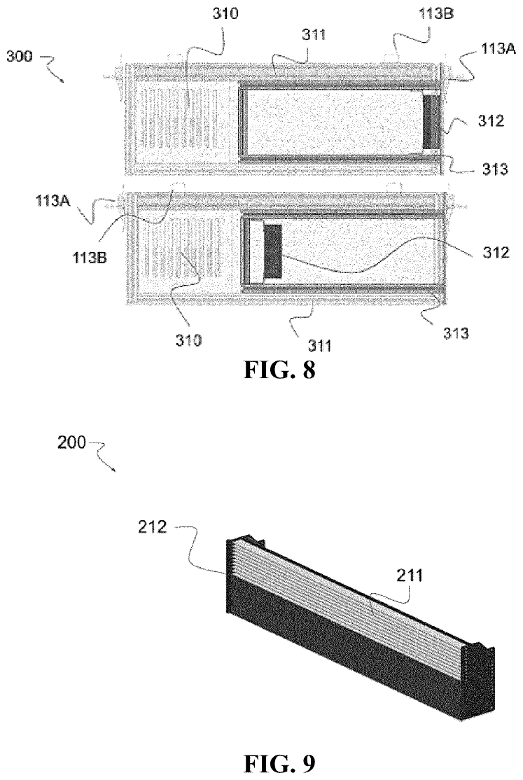

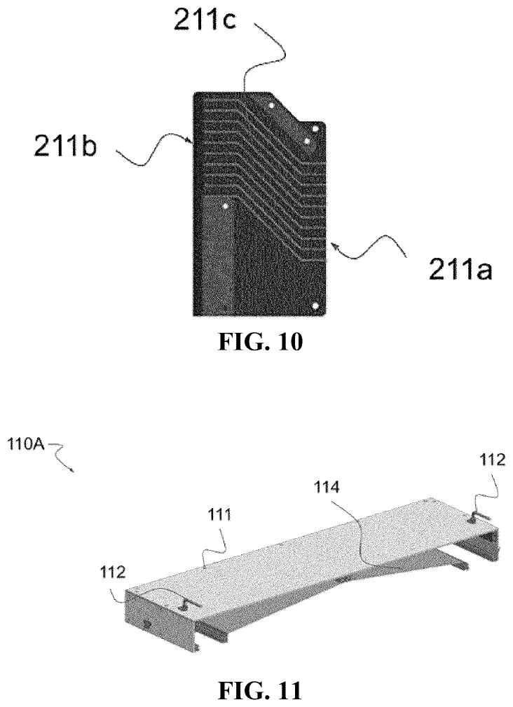

FIG. 1 is a schematic view showing the configuration of a multifunctional microbial unit cultivation apparatus according to an embodiment of the present invention, FIG. 2 is a schematic view showing the internal configuration of the cultivation module shown in FIG. 1, FIG. 3 is a perspective view of the cultivation module shown in FIG. 2, FIG. 4 is a perspective view of the cultivation module of FIG. 3 viewed from a different direction, FIG. 5 is a perspective view showing the internal configuration of the cultivation module shown in FIG. 2, FIG. 6 is a plan view of FIG. 5, FIG. 7 is a view for explaining the intake unit of the culture module shown in FIG. 5, FIG. 8 is a side view of FIG. 7 for explaining the movement structure of the blow fan, FIG. 9 is a perspective view of the eliminator shown in FIG. 5, FIG. 10 is a cross-sectional view of FIG. 9, and FIG. 11 is a view for explaining the configuration of the loop part provided in the upper case member among the cases shown in FIG. 3.

As illustrated in FIGS. 1 and 2, a multifunctional microbial unit cultivation apparatus (1) according to an embodiment of the present invention serves as a device for cultivating fungi, such as mushroom mycelium. The apparatus comprises a main module (10) that generates a mixed gas for microbial cultivation and controls the temperature and humidity of the mixed gas before supplying it. Additionally, the apparatus includes a cultivation module (100) connected to the main module (10), which utilizes the mixed gas supplied from the main module (10) for the cultivation of microorganisms. In FIG. 2, the directions of the arrows indicate the overall circulation direction of the mixed gas.

Here, the mixed gas can be circulated between the main module (10) and the cultivation module (100) by an intake structure.

The unit cultivation device (1) enables the provision of a mixed gas comprising carbon dioxide and oxygen to the cultivation section (400) at a regulated temperature and humidity. Specifically, the mixed gas undergoes appropriate humidification in accordance with the state of the cooled or heated gas, thereby preventing the occurrence of condensation within the cultivation module (100), where humidity and temperature control are executed. Moreover, this arrangement effectively inhibits the differentiation of mushrooms while promoting the growth of mycelium, which facilitates the formation of a fungal biomat.

Regarding each component, the main module (10) of this embodiment may include a housing (20) that forms the basic exterior, a gas mixing section (30) mounted on one side of the housing (20), a temperature control unit (40), and a humidity control section (50), as illustrated in FIGS. 1 and 2.

First, although not shown, the housing (20) may be provided with a humidification part including multiple pipes and a heat exchange part for heat exchange using flowing fluid.

The gas mixing unit (30) of the present embodiment, as illustrated in FIGS. 1 and 2, is configured to mix carbon dioxide and oxygen supplied from external sources to generate a mixed gas suitable for microbial cultivation in the cultivation unit (400).

The mixed gas must be provided to the cultivation module (100) after being adjusted to appropriate temperature and humidity levels. To facilitate this, the temperature control unit (40) of the present embodiment is capable of regulating the temperature of the mixed gas. As previously mentioned, the housing (20) is equipped with a heat exchange unit, wherein the temperature control unit (40) can control the operation of the heat exchange unit to regulate the temperature of the mixed gas.

Meanwhile, a water supply unit is connected to the humidity control unit (50) and can provide moisture to the mixed gas through interaction with the aforementioned humidification part, thereby controlling the humidity of the mixed gas.

In this manner, the main module (10) of the present embodiment can generate a mixed gas comprising carbon dioxide and oxygen. During this process, the humidity control unit (50) and the temperature control unit (40) work in conjunction to regulate the humidity and temperature of the mixed gas. Consequently, the mixed gas can be provided under optimal conditions for microbial cultivation in the cultivation unit (400) of the cultivation module (100), ensuring that microbial cultivation occurs within the most favorable environmental parameters.

Meanwhile, the cultivation module (100) of the present embodiment can be arranged in a stacked configuration with the main body module (10), as illustrated in FIG. 1. The main body module (10) and the cultivation module (100) are connected such that the mixed gas can be supplied from the main module (10) to the cultivation module (100). Additionally, the mixed gas can be recirculated from the cultivation module (100) back to the main module (10), facilitating a continuous flow of the mixed gas between the two modules.

The cultivation module (100) of the present embodiment facilitates the direct cultivation of microorganisms using the mixed gas provided from the main module (10). As illustrated in FIGS. 2 through 5, the cultivation module (100) comprises a casing member (110A, 110B), a cultivation unit (400) provided within the casing member (110A, 110B) for cultivating microorganisms, an intake unit (300) disposed on both sides of the cultivation unit (400) for delivering the mixed gas supplied from the main module (10) to the cultivation unit (400), and an eliminator (200) designed to prevent the dispersion of droplets mixed in the mixed gas.

First, the casing member (110A, 110B) of the present embodiment includes an upper casing member (110A) and a lower casing member (110B), as illustrated in FIGS. 2 through 6. The lower casing member (110B) can be equipped with the aforementioned components, including the cultivation unit (400), the intake unit (300), and the eliminator (200).

Referring to FIGS. 3 to 5, the lower casing member (110B) may have a rectangular parallelepiped shape open at the top, and the upper casing member (110A) may have a rectangular parallelepiped shape corresponding to the shape of the lower casing member (110B) and open at the bottom.

At the interface between the upper casing member (110A) and the lower casing member (110B), a locking mechanism may be employed, which includes a locking member (113A) having a hook (113B) and a corresponding engagement member. Specifically, the lower casing member (110B) is equipped with a hook-type locking member (113A), while the upper casing member (110A) is provided with an engagement member that is compatible with the locking member (113A). This configuration facilitates the efficient coupling and decoupling of the upper casing member (110A) with respect to the lower casing member (110B).

Additionally, as illustrated in FIGS. 3 and 4, the lower casing member (110B) and the upper casing member (110A) are equipped with multiple handle members (112). This configuration facilitates the movement of the casing member (110A, 110B) by the operator, enhancing operational convenience.

referring to FIGS. 3 and 4, the lower casing member (110B) and the upper casing member (110A) can maintain a sturdy configuration by the fastening of bolts (111) at the points where each side makes contact.

The cultivation unit (400) of the present embodiment, as illustrated in FIGS. 5 and 6, includes a plurality of cultivation vessels arranged in a parallel configuration. The incorporation of multiple cultivation vessels in this manner enhances the efficiency of microbial cultivation, allowing for a more effective use of space and resources within the cultivation module.

The intake unit (300) of the present embodiment, as depicted in FIGS. 2 and 5 through 8, is positioned on both sides of the cultivation unit (400) within the casing member (110A, 110B). The intake unit (300) may include a fan support structure (311) that forms a framework, a flow rate reduction member (310) that decreases airflow speed, a blow fan (312) for circulating air, and a rail member (313) that allows the blow fan (312) to be mounted in a movable manner.

As described above, a mixed gas having a predetermined temperature and humidity from the main module (10) is supplied to the cultivation unit (400) of the cultivation module (100) via the intake unit (300). The airflow speed is reduced by the flow rate reduction member (310), which minimizes the drying effect on the surface of the microorganisms during the microbial cultivation process.

Additionally, as referenced in FIGS. 7 and 8, the blow fan (312) may be mounted for linear movement on the rail member (313). Observing the upper view of FIG. 8, the blow fan (312) can be positioned at the right end of the rail member (313). As illustrated in the lower view, the blow fan (312) can traverse along the rail member (313) to be positioned at the left end. This allows for selective positional adjustment of the blow fan (312).

Meanwhile, the eliminator (200) of the present embodiment is provided on one side of the cultivation unit (400) inside the casing member (110A, 110B) as shown in FIGS. 5 and 6, and can prevent scattering of water droplets contained in the mixed gas.

This eliminator (200) may include, as shown in FIGS. 9 and 10, a support (212) and a scattering prevention unit (211) provided in a multi-layer structure on the support to prevent scattering of passing water droplets.

The scattering prevention unit (211) of this embodiment may include an inlet (211a) through which mixed gas provided from the body module (10) is introduced, an outlet (211b) for discharging the mixed gas provided through the inlet (211a) to the culture unit (400), and a connecting passage (211c) connecting the inlet (211a) and outlet (211b), where the outlet (211b) is located below the inlet (211a) so that the formation angle of the connecting passage (211b) can be 30 to 60 degrees.

More preferably, the formation angle of the connecting passage (211c) may be 45 degrees, so that when air containing water passes through the eliminator (200), large water droplet particles adhere to the inclined connecting passage (211c) while only air and water vapor in the air can pass through the eliminator (200).

Here, to maximize the area of the connecting passage (211c) of the eliminator (200) for the same area, the inlet (211a) and outlet (211b) may have a hexagonal structure. However, this is not limited, and it is natural that the inlet and outlet may have structures of other shapes.

To elaborate, in the conventional case, a method of indirectly humidifying by supplying water in fine particles was used, but in the present embodiment, humidification can be carried out by directly supplying air containing water vapor to the place to be air-conditioned. In other words, air and water can be continuously contacted in the eliminator (200) to increase the amount of water vapor contained in the air and supply it to the cultivation unit (400) of the cultivation module (100).

Therefore, unlike conventional centrifugal humidifiers or ultrasonic humidifiers, water can be supplied in the state of water vapor in the air, and through this, air containing moisture can be smoothly provided to the cultivation unit (400).

Meanwhile, referring to FIG. 11, a V-shaped loop (114) may be provided on the back surface of the upper casing member (110A) of this embodiment. This V-shaped loop (114) can prevent respiratory water generated during the respiration process of microorganisms from being collected and falling into the cultivation unit (400) where microorganisms are cultivated.

According to the present embodiment, a mixed gas containing carbon dioxide and oxygen can be provided to the cultivation unit (400) at an appropriate temperature and humidity. This enables humidity adjustment to proceed in accordance with the state of the cooled or heated mixed gas, thus preventing the occurrence of condensation within the cultivation module (100) where humidity and temperature control are implemented. Additionally, it can prevent the differentiation of mushrooms and facilitate the growth of mycelium, leading to the formation of a fungal biomat.

While specific embodiments of the present invention have been described thus far, various modifications can be made without departing from the scope of the invention.

Therefore, the scope of the invention should not be limited to the described embodiments, but rather should be defined by the claims that follow, as well as equivalents to those claims.

Although the present invention has been described with reference to the limited embodiments and drawings, the present invention is not limited to the above embodiments, and various modifications and variations are possible from this description by those skilled in the art to which the present invention pertains. Therefore, the spirit of the present invention should be understood only by the following patent claims, and all modifications or equivalent transformations thereof fall within the scope of the present invention.
1: Multifunctional microbial unit culture apparatus
10: main module
20: Housing
30: Gas mixing unit
40: Temperature control unit
50: Humidity control unit
100: Cultivation module
110A: Upper casing member
110B: Lower casing member
111: Bolt
112: Handle member
113A: engagement structure of the locking member and the engaged member
113B: Hook
114: Loop
200: Eliminator
211: Scattering prevention unit
211a: Inlet
211b: Outlet
211c: Connecting passage
300: Intake unit
310: Wind speed reduction member
311: Fan support
312: Blow fan
313: Rail member
400: Cultivation unit

### INDUSTRIAL APPLICABILITY

The present invention provides a mixed gas containing oxygen at an appropriate temperature and humidity to the cultivation unit. This enables the humidification of the cooled or heated mixed gas to proceed appropriately, thereby controlling the humidity and temperature within the cultivation module and preventing the occurrence of condensation. Additionally, it helps prevent the differentiation of mushrooms, making it applicable in microbial cultivation devices, thus demonstrating industrial applicability.

1. A multifunctional microbial unit culture apparatus comprising:
a main module that generates a mixed gas by mixing carbon dioxide and oxygen and controls the temperature and humidity of the mixed gas; and
a cultivation module having a cultivation unit that cultures microorganisms using the mixed gas supplied from the body module;
wherein the mixed gas circulates between the main module and the culture module.

2. The multifunctional microbial unit culture apparatus of claim 1, wherein the min module comprises:
a housing having a humidification unit including multiple pipes and a heat exchange unit for heat exchange using flowing fluid;
a gas mixing unit mounted on the housing that generates the mixed gas by mixing carbon dioxide and oxygen;
a temperature control unit mounted on the housing that controls the temperature of the mixed gas; and
a humidity control unit mounted on the housing that controls the humidity of the mixed gas,
wherein the mixed gas with temperature and humidity controlled by the temperature control unit and humidity control unit is provided to the culture module.

3. The multifunctional microbial unit culture apparatus of claim 2,
wherein the min module and the cultivation module have a stacked structure.

4. The multifunctional microbial unit culture apparatus of claim 1, wherein the cultivation module further comprises:
a case; and
an intake unit provided in the case for sending the mixed gas provided from the main module to the cultivation unit.

5. The multifunctional microbial unit culture apparatus of claim 4,
wherein the intake unit is disposed on both sides of the cultivation unit in the case,
and the intake unit comprises:
a wind speed reduction member for reducing wind speed;
a blow fan for circulating air; and
a rail member on which the blow fan is movably mounted.

6. The multifunctional microbial unit culture apparatus of claim 4,
wherein the cultivation module further comprises an eliminator provided on one side of the cultivation unit in the case to prevent scattering of water droplets contained in the mixed gas.

7. The multifunctional microbial unit culture apparatus of claim 6,
wherein the eliminator comprises:
a support fixed in the case; and
a scattering prevention unit provided in a multi-layer structure on the support to prevent scattering of passing water droplets.

8. The multifunctional microbial unit culture apparatus of claim 7,
wherein the scattering prevention unit includes an inlet through which the mixed gas provided from the main module is introduced, an outlet for discharging the mixed gas provided to the inlet to the culture unit, and a connecting passage connecting the inlet and the outlet,
wherein the outlet is positioned below the inlet such that the formation angle of the connecting passage is 30 to 60 degrees.

9. The multifunctional microbial unit culture apparatus of claim 8,
wherein the inlet and the outlet are arranged in a hexagonal configuration.

10. The multifunctional microbial unit culture apparatus of claim 4,
wherein the case comprises:
a lower casing member; and
an upper casing member detachably coupled to the lower casing member,
wherein one of the lower casing member and the upper casing member is provided with a locking member and the other is provided with a engaged member, such that coupling or separation of the upper casing member to/from the lower casing member is achieved by engagement or disengagement of the looking member and the engaged member.

11. The multifunctional microbial unit culture apparatus of claim 10,
wherein a V-shaped loop for collecting respiratory water generated during respiration of the microorganisms is provided on the back of the upper case member.

12. The multifunctional microbial unit culture apparatus of claim 1,
wherein the cultivation unit comprises multiple cultivation vessels arranged in a parallel structure.
